(19) United States
(12) Patent Application Publication
(10) Pub. No.: US 2024/0285660 A1
(43) Pub. Date: Aug. 29, 2024

(54) COMPOSITION FOR WOUND RELIEF AND INHIBITING A PHD PROTEIN AND METHODS OF TREATMENT

(71) Applicant: Curapep LLC, Burlington, WI (US)

(72) Inventors: Abdulhafez Selim, Burlington, WI (US); Amor Khachemoune, Burlington, WI (US); Lawrence Shimp, Burlington, WI (US)

(73) Assignee: Curapep LLC, Burlington, WI (US)

(21) Appl. No.: 18/572,641

(22) PCT Filed: Apr. 27, 2022
(86) PCT No.: PCT/US2022/026500

§ 371 Date: Dec. 20, 2023

Related U.S. Application Data
(60) Provisional application No. 63/201,379, filed on Apr. 27, 2021.

(51) Int. Cl. A61K 31/7048; A61K 9/06; A61K 31/352; A61K 47/10; A61K 47/20; A61K 47/44

(52) U.S. Cl. CPC A61K 31/7048; A61K 9/06; A61K 31/352; A61K 47/10; A61K 47/20; A61K 47/44

(57) ABSTRACT

Composition for administration to a subject comprising (a) an effective amount of a von Hippel Lindau protein inhibitor selected from meisoindigo, tanshinone IIA, 7-hydroxyflavone, dicumarol, flavone, tabersonine, danthron, equol, and pharmaceutically acceptable salts of each of the foregoing, and (b) an effective amount of a PHD2/EGLN1 protein inhibitor selected from glabridin, puerarin, wedelolactone, phlorizin and pharmaceutically acceptable salts of each the foregoing.

### FIELD

The presently disclosed subject matter relates to compositions containing a von Hippel Lindau protein inhibitor or an ARG-383 targeting component; and a PHD2/EGLN1 protein inhibitor or a HIS-313 targeting component; and methods of treatment that include administering these compositions to a subject.

### BACKGROUND

Cutaneous wounds resulting from conditions such as trauma, burns, surgery, and diabetes are common clinical problems. Following an acute injury, the skin wound microenvironment becomes more hypoxic due to vascular disruption and high oxygen consumption by the cells at the edges of the wound. This acute hypoxia plays a positive role in the early stages of wound healing, which induces the production of numerous cytokines in the wound microenvironment. Such as, for example, in the case of chronic wounds, an impaired response to tissue hypoxia is believed to be a major factor responsible for diminished wound healing. There remains a need for effective treatment for such conditions, and other conditions, and particularly treatment options involving natural products without significant adverse side effects.

### BRIEF SUMMARY

One embodiment of the presently disclosed subject matter provides a composition for administration to a subject comprising (a) an effective amount of a von Hippel Lindau protein inhibitor selected from meisoindigo, tanshinone IIA, 7-hydroxyflavone, dicumarol, flavone, tabersonine, danthron, equol, and an effective amount of a PHD2/EGLN1 protein inhibitor selected from glabridin, puerarin, wedelolactone, phlorizin.

In certain exemplary embodiments, the weight ratio of von Hippel Lindau protein inhibitor (e.g., flavone): PHD2/EGLN1 protein inhibitor (e.g., puerarin) is from about 0.01 to about 100, or from about 0.01 to about 0.5, or from about 0.5 to about 1.5.

Another embodiment of the presently disclosed subject matter provides a composition that includes (a) a first ARG-383 targeting component selected from flavone, echinatin, danthron, eriodictyol, caffeic acid phenethyl ester, myricetin, and xanthotoxol and (b) a second HIS-313 targeting component selected from puerarin, glabridin, wedelolactone, meisoindigo, sophoricoside, isovitexin and phlorizin.

Another embodiment of the presently disclosed subject matter provides a composition that includes a first ARG-383 targeting component (e.g., flavone) and/or a second HIS-313 targeting component (e.g., puerarin), such as a composition that includes an amount of flavone and an amount of peurarin. In exemplary embodiments, the composition contains a weight ratio of ARG-383 targeting component:HIS-313 targeting component or flavone:puerarin of from about 0.01 to about 100, or from about 0.01 to about 0.5, or from about 0.5 to about 1.5. In exemplary embodiments, the total weight percentage of ARG-383 targeting component (e.g., flavone) and HIS-313 targeting component (e.g., puerarin) is from about 0.1 to about 1.0, based on the total weight of the composition, though other relative and total amounts can find use according to the disclosed subject matter.

In an exemplary embodiment, the composition further includes a carrier, such as, but not limited to, one or more of petrolatum, mineral oil, corn oil, vegetable oil, glycerin, polyethylene glycol, sesame oil, coconut oil, olive oil, grapeseed oil, shea butter and olive butter. In another exemplary embodiment, the composition further includes a skin penetration enhancer and is in a form suitable for topical administration to the skin of an animal (e.g., a mammal, such as a human). Alternatively, in other embodiments, the composition is in a form for oral administration, or parenteral (e.g., subcutaneous, intraperitoneal or intravenous) administration to an animal (e.g., a mammal, such as a human), or is in a form for intragrastric delivery, or is in the form of a medical device (e.g., a bandage or implanted strand or other device).

Another embodiment of the presently disclosed subject matter provides a method of inhibiting a PHD protein (e.g., PHD2 protein) comprising administering to the subject an effective amount of a composition of the present disclosure. For example, the instantly disclosed compositions can be used in methods for promoting wound healing in a subject, methods of preventing or treating an inflammatory disease or condition in a subject, treating anemia in a subject, methods of regenerating and/or repairing cells, tissues or organs, and treating an ischemic disease or condition in a subject, that include administering to the subject an effective amount of a composition comprising a von Hippel Lindau protein inhibitor or an ARG-383 targeting component; and a PHD2/EGLN1 protein inhibitor or a HIS-313 targeting component, such as any one of the instantly disclosed compositions.

The presently disclosed subject matter is further directed to the general and specific embodiments defined, respectively, by the claims appended hereto, which are incorporated by reference herein.

### BRIEF DESCRIPTION OF THE DRAWINGS

The following detailed description of exemplary embodiments of the subject disclosure will be better understood when read in conjunction with the appended drawings. For the purpose of illustrating the present disclosure, exemplary embodiments and data from exemplary embodiments are shown in the drawings. It should be understood, however, that the subject application is not limited to the precise arrangements and instrumentalities shown.

FIG. 1 depicts the results of Boxplot is displaying HIF content data as a percent of the negative control as described in Example 3.

FIG. 2 depicts the probability of complete wound closure (CWC) over 20 days of treatment, as described in Example 4.

FIG. 3 depicts the probability of complete wound closure (CWC) on the 21^{st} day of treatment, as described in Example 4.

FIG. 4 depicts percentage of granulation tissue formation (GTSC) over 12 days of treatment, as described in Example 4.

### DETAILED DESCRIPTION

The disclosed subject matter can be more fully appreciated by reference to the following description, including the examples. Unless otherwise defined, all technical and scientific terms used herein have the same meaning as commonly understood by one of ordinary skill in the art. Although methods and materials similar or equivalent to those described herein can be used in the practice or testing of the presently disclosed subject matter, suitable methods and materials are described herein. In addition, the materials, methods, and examples are illustrative only and not intended to be limiting.

For the sake of brevity, all publications, including patent applications, patents, and other citations mentioned herein, are incorporated by reference in their entirety. Citation of any such publication, however, shall not be construed as an admission that it is prior art to the presently disclosed subject matter.

### Terms and Definitions

As used herein, the term “about” or “approximately” means within an acceptable range for a particular value as determined by one skilled in the art, and may depend in part on how the value is measured or determined, e.g., the limitations of the measurement system or technique. For example. “about” can mean a range of up to 20%, up to 10%, up to 5%, or up to 1% or less on either side of a given value. Alternatively, with respect to biological systems or processes, the term “about” can mean within an order of magnitude, within 5-fold, or within 2-fold on either side of a value. Numerical quantities given herein are approximate unless stated otherwise, meaning that the term “about” or “approximately” can be inferred when not expressly stated.

To provide a more concise description, some of the quantitative expressions given herein are not qualified with the term “about.” It is understood that, whether the term “about” is used explicitly or not, every quantity given herein is meant to refer to both the actual given value and the approximation of such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity for which that could be obtained under the particular stoichiometric conditions. Concentrations that are given as percentages refer to mass ratios, unless indicated differently.

As used herein, the terms “a,” “an,” and “the” are to be understood as meaning both singular and plural, unless explicitly stated otherwise. Thus, “a,” “an,” and “the” (and grammatical variations thereof where appropriate) refer to one or more.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosed subject matter may be described or claimed in the singular, the plural is contemplated to be within the scope thereof, unless limitation to the singular is explicitly stated.

The terms “comprising” and “including” are used herein in their open, non-limiting sense. Other terms and phrases used in this document, and variations thereof, unless otherwise expressly stated, should be construed as open ended, as opposed to limiting. Thus, the term “example” is used to provide exemplary instances of the item in discussion, not an exhaustive or limiting list thereof. Similarly, adjectives such as “conventional,” “traditional,” “normal,” “criterion,” “known,” and terms of similar meaning should not be construed as limiting the item described to a given time period or to an item available as of a given time, but they should be read to encompass conventional, traditional, normal, or criterion technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. As will become apparent to one of ordinary skill in the art after reading this document, the illustrated embodiments and their various alternatives may be implemented without confinement to the illustrated examples.

The term “composition” is intended to encompass a product including the herein described extracts and the inert ingredient(s) (pharmaceutically acceptable excipients) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. In certain embodiments, a “composition,” as used herein, is pharmaceutically acceptable and suitable for administration to a human. In certain embodiments, such compositions are administered in vitro. In certain embodiments such compositions are administered in vivo.

The term “carrier” refers to an adjuvant, vehicle, or excipients, with which the compound is administered. In certain embodiments, the carrier is a solid carrier. Suitable pharmaceutical carriers include those described in Remington: The Science and Practice of Pharmacy, 21^{st} Ed., Lippincott Williams & Wilkins (2005).

The term “dosage form,” as used herein, is the form in which the dose is to be administered to the subject or patient. The instantly disclosed active agents are generally administered as part of a formulation that includes nonmedical agents. Dosage forms, for example, can be solid, liquid, gel or gaseous. In certain exemplary embodiments, the dosage form is a topical dosage form, and more specifically, comprises a gel, lotion or other form suitable for application to human skin. In other embodiments, the dosage form is suitable for oral, parenteral or other forms of administration.

The term “pharmaceutically acceptable,” as used in connection with compositions of the disclosed subject matter, refers to molecular entities and other ingredients of such compositions that are physiologically tolerable and do not typically produce untoward reactions when administered to an animal (e.g., human) according to their intended mode of administration (e.g., oral or topical).

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluents to facilitate administration of an agent and that is compatible therewith. Examples of excipients include, but are not limited to, dimethyl sulfoxide (DMSO), petrolatum, mineral oil, calcium carbonate, calcium phosphate, various sugars and types of starch, cellulose derivatives, gelatin, vegetable oils (e.g., corn oil), and polyethylene glycols. Suitable pharmaceutical carriers include those described in Remington: The Science and Practice of Pharmacy, 21V Ed., Lippincott Williams & Wilkins (2005).

As used herein, the term “inert” refer to any inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

As used herein, “suitable for oral administration” or “suitable for topical administration” or “suitable for administration to a subject” refers to a sterile, pharmaceutical product, such as a product produced under good manufacturing practices (GMP).

As used herein, the term “disorder” is used interchangeably with “disease” or “condition”. For example, a neurological disorder also means a neurological disease or a neurological condition.

The terms “treat,” “treating.” and “treatment” cover therapeutic methods directed to a disease-state in a subject and include: (i) preventing the disease-state from occurring, in particular, when the subject is predisposed to the disease-state but has not yet been diagnosed as having it; (ii) inhibiting the disease-state, e.g., arresting its development (progression) or delaying its onset; and (iii) relieving the disease-state, e.g., causing regression of the disease state until a desired endpoint is reached. These terms also include ameliorating a symptom of a disease (e.g., reducing the pain, discomfort, or deficit), wherein such amelioration may be directly affecting the disease (e.g., affecting the disease's cause, transmission, or expression) or not directly affecting the disease.

As used in the present disclosure, the term “effective amount” is interchangeable with “therapeutically effective amount” and means an amount or dose of active ingredient effective in treating the particular disease, condition, or disorder disclosed herein, and thus “treating” includes producing a desired preventative, inhibitory, relieving, or ameliorative effect. In methods of treatment according to the disclosed subject matter, “an effective amount” of at least one compound is administered to a subject (e.g., a mammal). The “effective amount” will vary, depending on the compound, the disease (and its severity), the treatment desired, age and weight of the subject, etc.

As used herein, the phrase “in combination” refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule: it is not required that individual doses of different agents be administered at the same time, or in the same composition. Rather, so long as both (or more) agents remain in the subject's body, they are considered to be administered “in combination”.

The terms “individual,” “subject,” and “patient” are used interchangeably herein and can be a vertebrate, in particular, a mammal, more particularly, a primate (including non-human primates and humans) and include a laboratory animal in the context of a clinical trial or screening or activity experiment. Thus, as can be readily understood by one of ordinary skill in the art, the instantly disclosed compositions and methods are particularly suited to administration to any vertebrate, particularly a mammal, and more particularly, a human.

The concentration of the substance is selected so as to exert its therapeutic effect, but low enough to avoid significant side effects is within the scope and sound judgment of the skilled artisan. The effective amount of the composition can vary with the age and physical condition of the biological subject being treated, the severity of the condition, the duration of the treatment, the nature of concurrent therapy, the specific compound, composition or other active ingredient employed, the particular carrier utilized, and like factors. Those of skill in the art can readily evaluate such factors and, based on this information, determine the particular effective concentration of a composition of the presently disclosed subject matter to be used for an intended purpose.

Reference will now be made to the embodiments of the presently disclosed subject matter, examples of which are illustrated by and described in conjunction with the accompanying examples. While certain embodiments are described herein, it is understood that the described embodiments are not intended to limit the scope of the presently disclosed subject matter. On the contrary, the present disclosure is intended to cover alternatives, modifications, and equivalents that can be included within the presently disclosed subject matter as defined by the claims.

The dosage for treatment is administration, by any of the foregoing means or any other means known in the art, of an amount sufficient to bring about the desired therapeutic effect. Thus, an effective amount includes an amount of a provided composition (or mixture of provided compositions) or pharmaceutical composition of the disclosed subject matter that is sufficient to induce a desired effect, including a wound healing effect.

### PHD2/EGLN1 Protein Inhibitors

Stabilization of HIF1-α under normoxia results from the inability to hydroxylate HIF-α (which is mediated by PHD) or to efficiently ubiquitylate hydroxylated HIF-α (which is mediated by VHL). See. e.g., Volker H. Haase, The VHL Tumor Suppressor: Master Regulator of HIF; Curr. Pharm Des. 2009; 15(33):3895-3903. Pharmacological stabilization of HIF-α offers enormous opportunities for the treatment of various conditions, including, for example, anemia and ischemic disorders and tissues regeneration.

Embodiments of the subject disclosure stabilize HIF-α by concomitantly targeting VHL and PHD proteins. Accordingly, in exemplary embodiments, the composition includes (a) a PHD2/EGLN1 protein inhibitor and (b) a von Hippel Lindau protein inhibitor.

In one exemplary embodiment, the PHD2/EGLN1 protein inhibitor is selected from the group consisting of glabridin, puerarin, wedolactone and phlorizin.

### Von Hippel Lindau Protein Inhibitors

In one exemplary embodiment, the von Hippel Lindau protein (VHLP) inhibitor is selected from the group consisting of mesoindigo, tanshinone IIA (Tan IIA), 7-hydroxyflavone, dicumarol, flavone, tabersonine, danthron, and equol ((3S)-3-(4-Hydroxyphenyl)-3,4-dihydro-2H-1-benzopyran-7-ol).

In one embodiment, the instantly disclosed composition includes any one or more PHD2/EGLN1 protein inhibitor, such as, but not limited to, the those PHD2/EGLN1 inhibitors specifically disclosed herein, and any one or more VHLP inhibitor, such as, but not limited to, those VHLP inhibitors specifically disclosed herein. For the sake of brevity, reference to a PHD2/EGLN1 protein inhibitor throughout this application should be construed to be a reference to any one of the PHD2/EGLN1 protein inhibitors disclosed herein. Likewise, reference to a von Hippel Lindau protein inhibitor or a VHLP inhibitor throughout this application should be construed to be a reference to any one of the von Hippel Lindau protein VHLP inhibitors disclosed herein.

In one embodiment, the composition includes glabridin and meisoindigo. In one embodiment, the composition includes glabridin and tanshinone IIA. In one embodiment, the composition includes glabridin and 7-hydroxyflavone. In one embodiment, the composition includes glabridin and dicumarol. In one embodiment, the composition includes glabridin and flavone. In one embodiment, the composition includes glabridin and tabersonine. In one embodiment, the composition includes glabridin and danthron. In one embodiment, the composition includes glabridin and equol.

In one embodiment, the composition includes puerarin and meisoindigo. In one embodiment, the composition includes puerarin and tanshinone IIA. In one embodiment, the composition includes puerarin and 7-hydroxyflavone. In one embodiment, the composition includes puerarin and dicumarol. In one embodiment, the composition includes puerarin and flavone. In one embodiment, the composition includes puerarin and tabersonine. In one embodiment, the composition includes puerarin and danthron. In one embodiment, the composition includes puerarin and equol.

In one embodiment, the composition includes wedelolactone and meisoindigo. In one embodiment, the composition includes wedelolactone and tanshinone IIA. In one embodiment, the composition includes wedelolactone and 7-hydroxyflavone. In one embodiment, the composition includes wedelolactone and dicumarol. In one embodiment, the composition includes wedelolactone and flavone. In one embodiment, the composition includes wedelolactone and tabersonine. In one embodiment, the composition includes wedelolactone and danthron. In one embodiment, the composition includes wedelolactone and equol.

In one embodiment, the composition includes phlorizin and meisoindigo. In one embodiment, the composition includes phlorizin and tanshinone IIA. In one embodiment, the composition includes phlorizin and 7-hydroxyflavone. In one embodiment, the composition includes phlorizin and dicumarol. In one embodiment, the composition includes phlorizin and flavone. In one embodiment, the composition includes phlorizin and tabersonine. In one embodiment, the composition includes phlorizin and danthron. In one embodiment, the composition includes phlorizin and equol.

### ARG-383 Targeting Components

As explained in Example 1, according to one exemplary embodiment, structure key residues or drug pocket locations were identified at key residues of His 313 and Arg 383 on the PHD2 protein domain. Accordingly, in exemplary embodiments of the subject disclosure, the composition includes (a) an ARG-383 targeting component (e.g., flavone) and (b) a HIS-313 targeting component (e.g., puerarin).

Flavone (CAS RN 525-82-6), as used herein, refers to 2-phenylchromen-4-one (IUPAC), having a molecular weight of 222.24 g/mol and the molecular formula of C₁₅H₁₀O₂, and includes pharmaceutically acceptable salts thereof. Flavone can be obtained commercially as a synthesized compound, or prepared synthetically according to known methods. In such embodiments, the flavone can be introduced, for example at a purity of at least 90 wt %, 95 wt %, 97.5 wt %, 98 wt %, or higher.

In certain embodiments the ARG-383 targeting component (e.g., flavone) can be obtained from an extract. Alternatively, in other embodiments, the flavone is not obtained from an extract, and/or the flavone is prepared synthetically.

Other ARG-383 targeting components (i.e., echinatin [CAS No. 525-82-6], danthron [CAS No. 97207-47-1], eriodictyol [CAS No. 552-58-9], caffeic acid phenethyl ester [CAS No. 104594-70-9], myricetin [CAS No. 529-44-2], and xanthotoxol [CAS No. 2009-24-7]) can similarly be obtained from commercial sources and/or synthetically prepared. These active agents can also be obtained from their botanical or food source (e.g., as included within an extract of the botanical or food source).

### HIS-313 Targeting Components

Puerarin (CAS RN 3681-99-0), as used herein, refers to 7-hydroxy-3-(4-hydroxyphenyl)-8-[(2S,3R,4R,5S,6R)-3,4,5-trihydroxy-6-(hydroxymethyl)oxan-2-yl]chromen-4-one (IUPAC), having a molecular weight of 416.4 g/mol and the molecular formula of C₂₁H₂₀O₉, and includes pharmaceutically acceptable salts thereof. The HIS-313 Targeting Component (e.g., puerarin) can be obtained commercially as a synthesized compound, or prepared synthetically according to known methods. In such embodiments, the HIS-313 Targeting Component can be introduced, for example at a purity of at least 90 wt %, 95 wt %, 97.5 wt %, 98 wt %, or higher.

In certain embodiments HIS-313 Targeting Component (e.g., puerarin) can be obtained from an extract, such as, for example, an extract obtained from Radix Pueraria lobata (RP), the dried root of Pueraria lobata (willd.). See, e.g., Hyo Won Jung et al., Nutrients, 2017, 9, 33. Alternatively, in other embodiments, the HIS-313 Targeting Component (e.g., puerarin) is not obtained from an extract, and/or the HIS-313 Targeting Component (e.g., puerarin) is prepared synthetically.

Other HIS-313 targeting components (i.e., glabridin [CAS No. 59870-68-7], wedelolactone [CAS No. 524-12-9], meisoindigo [CAS No. 97204-47-1], sophoricoside [CAS No. 152-95-4], isovitexin [CAS No. 38953-85-4] and phlorizin [CAS No. 60-81-1]) can similarly be obtained from commercial sources and/or synthetically prepared. These active agents can also be obtained from their botanical or food source (e.g. as included within an extract of the botanical or food source).

In one embodiment, the composition includes any one or more ARG-383 targeting component, such as, but not limited to, those ARG-383 targeting components specifically disclosed herein, and any one or more HIS-313 targeting component, such as, but not limited to, those HIS-313 targeting components specifically disclosed herein. For the sake of brevity, reference to an ARG-383 targeting component throughout this application should be construed to be a reference to any one of the ARG-383 targeting components disclosed herein. Likewise, reference to a HIS-313 targeting component throughout this application should be construed to be a reference to any one of the HIS-313 targeting components disclosed herein.

In one embodiment, the composition includes flavone and puerarin. In one embodiment, the composition includes echinatin and puerarin. In one embodiment, the composition includes danthron and puerarin. In one embodiment, the composition includes eriodictyol and puerarin. In one embodiment, the composition includes caffeic acid phenethyl ester and puerarin. In one embodiment, the composition includes myricetin and puerarin. In one embodiment, the composition includes xanthotoxol and puerarin.

In one embodiment, the composition includes flavone and glabridin. In one embodiment, the composition includes echinatin and glabridin. In one embodiment, the composition includes danthron and glabridin. In one embodiment, the composition includes eriodictyol and glabridin. In one embodiment, the composition includes caffeic acid phenethyl ester and glabridin. In one embodiment, the composition includes myricetin and glabridin. In one embodiment, the composition includes xanthotoxol and glabridin.

In one embodiment, the composition includes flavone and wedelolactone. In one embodiment, the composition includes echinatin and wedelolactone. In one embodiment, the composition includes danthron and wedelolactone. In one embodiment, the composition includes eriodictyol and wedelolactone. In one embodiment, the composition includes caffeic acid phenethyl ester and wedelolactone. In one embodiment, the composition includes myricetin and wedelolactone. In one embodiment, the composition includes xanthotoxol and wedelolactone.

In one embodiment, the composition includes flavone and meisoindigo. In one embodiment, the composition includes echinatin and meisoindigo. In one embodiment, the composition includes danthron and meisoindigo. In one embodiment, the composition includes eriodictyol and meisoindigo. In one embodiment, the composition includes caffeic acid phenethyl ester and meisoindigo. In one embodiment, the composition includes myricetin and meisoindigo. In one embodiment, the composition includes xanthotoxol and meisoindigo.

In one embodiment, the composition includes flavone and sophoricoside. In one embodiment, the composition includes echinatin and sophoricoside. In one embodiment, the composition includes danthron and sophoricoside. In one embodiment, the composition includes eriodictyol and sophoricoside. In one embodiment, the composition includes caffeic acid phenethyl ester and sophoricoside. In one embodiment, the composition includes myricetin and sophoricoside. In one embodiment, the composition includes xanthotoxol and sophoricoside.

In one embodiment, the composition includes flavone and isovitexin. In one embodiment, the composition includes echinatin and isovitexin. In one embodiment, the composition includes danthron and isovitexin. In one embodiment, the composition includes eriodictyol and isovitexin. In one embodiment, the composition includes caffeic acid phenethyl ester and isovitexin. In one embodiment, the composition includes myricetin and isovitexin. In one embodiment, the composition includes xanthotoxol and isovitexin.

In one embodiment, the composition includes flavone and phlorizin. In one embodiment, the composition includes echinatin and phlorizin. In one embodiment, the composition includes danthron and phlorizin. In one embodiment, the composition includes eriodictyol and phlorizin. In one embodiment, the composition includes caffeic acid phenethyl ester and phlorizin. In one embodiment, the composition includes myricetin and phlorizin. In one embodiment, the composition includes xanthotoxol and phlorizin.

### Compositions

All active components disclosed herein (e.g., von Hippel Lindau protein (VHLP) inhibitor, PHD2/EGLN1 protein inhibitor, ARG-383 targeting component, and HIS-313 targeting component) can be obtained from botanical sources, or can also be commercially obtained as synthesized molecules, or synthesized by one of ordinary skill in the art.

In certain embodiments, the compositions of the present application contain a weight ratio of von Hippel Lindau protein (VHLP) inhibitor:PHD2/EGLN1 protein inhibitor of from about 0.01 to about 100, or from about 0.05 to about 50, or from about 0.1 to about 10, or from about 0.5 to about 1.5, or from about 0.75 to about 1.25 (e.g., 1.0).

In certain exemplary embodiments, a PHD2/EGLN1 protein inhibitor rich composition is preferred, such the weight ratio of von Hippel Lindau protein (VHLP) inhibitor:PHD2/EGLN1 protein inhibitor is, for example, from about 0.001 to about 0.9, or about 0.01 to about 0.75, or from about 0.05 to about 0.5, or from about 0.1 to 0.4, or from about 0.2 to 0.3 (e.g., 0.25).

In certain exemplary embodiments, a composition with equal or substantially equal amounts of von Hippel Lindau protein (VHLP) inhibitor and PHD2/EGLN1 protein inhibitor is preferred, such the weight ratio of von Hippel Lindau protein (VHLP) inhibitor:PHD2/EGLN1 protein inhibitor is, for example, from about 0.7 to about 1.3, or about 0.8 to about 1.2, or from about 0.9 to about 1.1 (e.g., 1.0).

In certain embodiments the total weight percentage of PHD2/EGLN1 protein inhibitor and von Hippel Lindau protein (VHLP) inhibitor together in the composition is from about 0.01 wt % to about 10 wt %, or from about 0.1 wt % to about 1.0 wt %, or from about 0.25 wt % to about 0.75 wt % (e.g., 0.5 wt %), based on the total weight of the composition. Such weight percentages can find use, for example, in topical formulations and can be modified accordingly depending on the desired route of administration.

In certain embodiments the total weight of PHD2/EGLN1 protein inhibitor in the composition is from about 1 mg/kg to about 160 mg/kg, or from about 2 mg/kg to about 80 mg/kg, or from about 4 mg/kg to about 40 mg/kg, based on the weight of the subject and/or the total weight of the von Hippel Lindau protein (VHLP) inhibitor in the composition is from about 0.625 mg/kg to about 100 mg/kg, or from about 1.25 mg/kg to about 50 mg/kg, or from about 2.5 mg/kg to about 25 mg/kg, based on the weight of the subject. Such weight amounts can find use, for example, in systemic formulations, as a unit dose.

In certain embodiments, the compositions of the present application contain a weight ratio of ARG-383 targeting component:HIS-313 targeting component of from about 0.01 to about 100, or from about 0.05 to about 50, or from about 0.1 to about 10, or from about 0.5 to about 1.5, or from about 0.75 to about 1.25 (e.g., 1.0).

In certain embodiments, the compositions of the present application contain a weight ratio of ARG-383 targeting component:HIS-313 targeting component of from about 0.01 to about 1, or from about 0.05 to about 0.75, or from about 0.1 to about 0.5, or from about 0.2 to about 0.3 (e.g., 0.25).

In certain exemplary embodiments, a HIS-313 targeting component rich composition is preferred, such the weight ratio of ARG-383 targeting component:HIS-313 targeting component is, for example, from about 0.001 to about 0.9, or about 0.01 to about 0.75, or from about 0.05 to about 0.5, or from about 0.1 to 0.4, or from about 0.2 to 0.3 (e.g., 0.25).

In certain exemplary embodiments, a composition with equal or substantially equal amounts of ARG-383 targeting component and HIS-313 targeting component is preferred, such the weight ratio of ARG-383 targeting component:HIS-313 targeting component is, for example, from about 0.7 to about 1.3, or about 0.8 to about 1.2, or from about 0.9 to about 1.1 (e.g., 1.0).

In certain embodiments, the compositions of the present application contain a weight ratio of flavone:puerarin of from about 0.01 to about 100, or from about 0.05 to about 50, or from about 0.1 to about 10, or from about 0.5 to about 1.5, or from about 0.75 to about 1.25 (e.g., 1.0).

In certain embodiments, the compositions of the present application contain a weight ratio of flavone:puerarin of from about 0.01 to about 1, or from about 0.05 to about 0.75, or from about 0.1 to about 0.5, or from about 0.2 to about 0.3 (e.g., 0.25).

In certain exemplary embodiments, a puerarin rich composition is preferred, such the weight ratio of flavone:puerarin is, for example, from about 0.001 to about 0.9, or about 0.01 to about 0.75, or from about 0.05 to about 0.5, or from about 0.1 to 0.4, or from about 0.2 to 0.3 (e.g., 0.25).

In certain exemplary embodiments, a composition with equal or substantially equal amounts of flavone and puerarin is preferred, such the weight ratio of flavone:puerarin is, for example, from about 0.7 to about 1.3, or about 0.8 to about 1.2, or from about 0.9 to about 1.1 (e.g., 1.0).

In certain embodiments the total weight percentage of ARG-383 targeting component and HIS-313 targeting component together in the composition is from about 0.01 to about 10, or from about 0.1 to about 1.0, or from about 0.25 to about 0.75 (e.g., 0.5 wt %), based on the total weight of the composition. Such weight percentages can find use, for example, in topical formulations and can be modified accordingly depending on the desired route of administration.

In certain embodiments the total weight percentage of flavone and puerarin together in the composition is from about 0.01 to about 10, or from about 0.1 to about 1.0, or from about 0.25 to about 0.75 (e.g., 0.5 wt %), based on the total weight of the composition. Such weight percentages can find use, for example, in topical formulations and can be modified accordingly depending on the desired route of administration.

In certain embodiments the total weight of ARG-383 targeting component in the composition is from about 1 mg/kg to about 160 mg/kg, or from about 2 mg/kg to about 80 mg/kg, or from about 4 mg/kg to about 40 mg/kg, based on the weight of the subject and/or the total weight of the HIS-313 targeting compound in the composition is from about 0.625 mg/kg to about 100 mg/kg, or from about 1.25 mg/kg to about 50 mg/kg, or from about 2.5 mg/kg to about 25 mg/kg, based on the weight of the subject. Such weight amounts can find use, for example, in systemic formulations, as a unit dose.

In certain embodiments the total weight of flavone in the composition is from about 1 mg/kg to about 160 mg/kg, or from about 2 mg/kg to about 80 mg/kg, or from about 4 mg/kg to about 40 mg/kg, based on the weight of the subject and/or the total weight of the puerarin in the composition is from about 0.625 mg/kg to about 100 mg/kg, or from about 1.25 mg/kg to about 50 mg/kg, or from about 2.5 mg/kg to about 25 mg/kg, based on the weight of the subject. Such weight amounts can find use, for example, in systemic formulations, as a unit dose.

Generally, the compositions of the present disclosure can be administered by any route of administration, including, but not limited to, oral, intravenous, intraperitoneal, intragastric, and topical forms of administration. The instantly disclosed compositions can also be incorporated into medical devices, including but not limited to medical devices to adhere to or reside in an area to be treated or that are implanted into the subject.

In certain embodiments, compositions suitable for oral administration are excluded, and methods of treating diseases or conditions that include oral administration of a compound as disclosed herein are excluded. In certain embodiments, compositions containing puerarin and not flavone are excluded. In certain exemplary embodiments, the composition consists essentially of meisoindigo, or a pharmaceutically acceptable salt thereof.

### Methods of Treatment

Hypoxia-inducible factor-1 (HIF-1), a heterodimeric transcription factor complex, has been recognized as a master regulator of hypoxia. HIF-1B protein levels remain relatively constant, while HIF-1α is hydroxylated on conserved proline residues under normoxic conditions, which results in von Hippel-Lindau (VHL) protein E3 ubiquitin ligase recruitment and immediate proteasomal degradation. This prolyl hydroxylation is mediated by an enzyme family of HIF-prolyl hydroxylases (prolyl-4-hydroxylation domain proteins (hereafter “PHD proteins”). The activity of PHD proteins are dependent on dioxygen, ferrous iron and 2-oxoglutarate.

One such PHD protein, PHD2 protein, is a 46 KDa enzyme encoded by the EGLN1 gene. PHD2 consists of an N-terminal domain homologous to MYND zinc finger domains, and a C-terminal domain homologous to the 2-oxoglutarate dioxygenases. The catalytic domain consists of a double-stranded i-helix core that is stabilized by three α-helices packed along the major β-sheet. See, e.g., McDonough et al., Proc Natl Acad Sci USA. 103(26): 9814-9, hereby incorporated by reference in its entirety. An active site, which is contained in the pocket between the β-sheets, chelates iron(II) through histidine and aspartate coordination. 2-oxoglutarate displaces a water molecule to bind iron as well. See Chowdhury et al, Structure 17 (7): 981-9 (2009), hereby incorporated by reference in its entirety. The active site is lined by hydrophobic residues, possibly because such residues are less susceptible to potential oxidative damage by reactive species leaking from the iron center. See MDonough et al., Proc Natl Acad Sci USA. 103(26): 9814-9.

PHD proteins are less active in hypoxic conditions, in which HIF-1a translocates to the nucleus and dimerizes with HIF-18, which allows the complex to bind to the hypoxia response elements (HREs) in the regulatory sequence of several genes vital to cell survival.

PHD inhibitors have been previously disclosed. FG-4592 is a PHD inhibitor is currently in phase-three clinical trials for the treatment of renal anemia inpatients with chronic kidney disease. FG-4592 has also been observed to improve EpSC proliferation and motility by stabilizing HIF-1α, thus suggesting that HIF-1α is an important target through which wound healing can be accelerated. See Cell Physiol Biochem 2018; 46:2460-2470, hereby incorporated by reference. In recent years, other studies have shown that novel small molecules can stabilize HIF-1α and increase tolerance to hypoxia, thereby improving the prognosis of many diseases and conditions, such as, but not limited to, renal anaemia, spinal cord injury and retinal disease.

With respect to wound healing, HIF-1α affects the wound-healing process in many ways, as it affects the expression of multiple angiogenic growth factors, cell motility, and the recruitment of endothelial progenitor cells, as well as the better local availability of erythropoietin and insulin. Recent studies using gene-knockout models have demonstrated that both PHD-2 and HIF-1α have an effect on wound healing.

In certain exemplary embodiments, the presently disclosed compositions are administered to a subject (e.g., topically or systemically administered to a human subject) to inhibit a PHD protein (e.g., PHD2 protein). In an exemplary embodiment, the presently disclosed compositions selectively inhibit PHD2 protein.

In certain exemplary embodiments, the presently disclosed compositions are administered to a subject (e.g., topically or systemically administered to a human subject) to promote wound healing. For example, the presently disclosed compositions can be administered to promote chronic wound healing (i.e., promoting healing chronic wounds), such as subjects who have chronic wounds as a result of diabetes or other comorbidity. The wound can be, for example, a wound (e.g., an acute wound) at risk of delayed healing, due either to the nature of the wound itself, or the age and/or comorbidities associated with the subject.

In certain exemplary embodiments, the presently disclosed compositions are administered to a subject to treat a skin ulcer. The skin ulcer can be, for example, a diabetic or neuropathic ulcer, a pressure ulcer (bed sore), a venous insufficiency ulcer, an arterial ulcer.

In certain exemplary embodiments, the presently disclosed compositions are administered to a subject (e.g., topically or systemically administered to a human subject) to treat or prevent scar formation (e.g., to prevent abnormal scar formation). For example, the presently disclosed compositions can be administered to a wound to prevent the formation of a scar and/or other surface or superficial blemishes as the skin as the wound heals. Accordingly, the presently disclosed compositions can be administered after a wound, including wounds induced or attendant to, dental, cosmetic or surgical procedures, to prevent the formation of a scar or blemish.

In certain exemplary embodiments, the presently disclosed compositions are administered to a subject (e.g., topically or systemically administered to a human subject) to treat an inflammatory disease or condition. For example, the presently disclosed compositions can be administered to treat an inflammatory disease or condition selected from allergy, asthma. COPD, autoimmune diseases, celiac disease, colitis, irritable bowel syndrome, intestinal hyperplasia, metabolic syndrome, obesity, diabetes, rheumatoid arthritis, liver disease, hapatic steatosis, fatty liver disease, non-alcoholic fatty liver disease, and non-alcoholic steatohepatitis, glomerulonephritis, hepatitis, inflammatory bowel disease, reperfusion injury and transplant rejection.

In certain exemplary embodiments, the presently disclosed compositions are administered to a subject (e.g., topically or systemically administered to a human subject) to treat anemia. For example, the presently disclosed compositions can be administered to treat an anemia selected from iron-deficiency anemia, an anemia caused by bone marrow and stem cell abnormalities, sickle cell anemia, vitamin-deficiency anemia, drug-induced immune hemolytic anemia, and anemias caused by other chronic conditions, such as advanced kidney disease (i.e., anemia of chronic kidney diseases), hypthothyroidism, old age and chronic diseases such as cancer, blood disorders (e.g., thrombotic thrombocytopenic purpura (TP) and thalassemia), infection, lupus, diabetes and rheumatoid arthritis.

In certain exemplary embodiments, the presently disclosed compositions are administered to a subject (e.g., topically or systemically administered to a human subject) to treat an ischemic disease, event or condition. For example, the presently disclosed compositions can be administered to treat an ischemic disease event or condition selected from atherosclerosis, trauma, ischemic cerebrovascular disorder (such as stroke or cerebral infarction), ischemic renal disease, ischemic pulmonary disease, infection-related ischemic disease, ischemic disease of limbs, and ischemic heart disease (such as ischemic cardiomyopathy, myocardial infarction, myocardial ischemia condition, or ischemic heart failure).

In certain exemplary embodiments, the presently disclosed compositions are administered to a subject (e.g., topically or systemically administered to a human subject) to treat a disorder or condition selected from hyperoxia-induced neonatal lung disease, Alzheimer's disease, prevention and treatment of organ fibrosis, bone fracture, chemotherapy-induced organ toxicity, osteoporosis, depression, and Parkinson's disease. In alternative embodiments, the presently disclosed compositions are used attendant to tissue engineering procedures (e.g., to regenerate and/or repair cells or tissues in connection with such procedures), and/or to provide one or more of a radioprotective effect, or a chemoprotective effect.

In certain exemplary embodiments, the presently disclosed compositions are administered to a subject (e.g., topically or systemically administered to a human subject) to regenerate and/or repair a cell, tissue or organ. In an exemplary embodiment, the cell, tissue or organ is selected from soft tissue, tendons, muscles, connective tissues, ligaments, tendons, cartilage, fibrocartilage, bone, neuronal tissue, vascular tissue, fascia, hematopoietic stem cells, liver, pancreas, kidney, brain, eye, mucous membrane, dermal, skin, epithelium, lung, pancreas, gastrointestinal, ocular tissue, gum tissue and dental tissue and teeth.

The presently disclosed compositions can be administered to human subjects. In alternative embodiments, the compositions are administered to non-human animal subjects, such as, but not limited to, cats, dogs, horses, cows, sheep, pigs, reptiles, rodents, monkeys; and zoo, performance, or other captive animals.

For topical administration formulations, any of a variety of creams, ointments, gels, cleansers, lotions and the like can be employed. For many pharmaceutical formulations, non-active ingredients will constitute the greater part, by weight or volume, of the preparation. For pharmaceutical formulations, it is also contemplated that any of a variety of measured-release, slow-release or time-release formulations and additives can be employed, so that the dosage can be formulated so as to effect delivery of a provided composition over a period of time. For example, gelatin, sodium carboxymethylcellulose and/or other cellulosic excipients can be included to provide time-release or slower-release formulations, especially for administration by subcutaneous and intramuscular injection. Other additives which can be used include petroleum jelly, lanoline, polyethylene glycols, alcohols, transdermal enhancers, and combinations of two or more thereof.

In certain exemplary embodiments, formulations suitable for topical application achieve transdermal delivery. Transdermal pharmaceutical devices include patches, occlusive dressings, occlusive formulations, hypodermic sprays, iontophoretic systems, gels and infusion pumps, all of which are well known in the art. A transdermal patch which includes a pharmaceutical can generally include a backing layer impermeable to the pharmaceutical, a reservoir to house the pharmaceutical, and an adhesive cover to be removed upon use of the patch and for adhesion to the skin of a patient.

Formulations suitable for transdermal administration can also be presented as medicated bandages or discrete patches adapted to remain in intimate contact with the epidermis of the recipient for a prolonged period of time. Representative examples of suitable transdermal patches include, for example, those developed by NeuroDerm Ltd (Israel) and/or that used to deliver estradiol, for example, those developed by Novogyne Pharmaceuticals. Formulations suitable for transdermal administration can also be delivered by iontophoresis (passage of a small electric current (15 mA) to “inject” electrically charged ions into the skin) through the skin. For this, the dosage form typically takes the form of an optionally buffered aqueous solution of the active composition.

The compositions of the presently disclosed subject matter can be incorporated into all types of vehicles. Non-limiting examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the presently disclosed subject matter. In certain aspects, it is important that the concentrations and combinations of the compounds, ingredients, and agents be selected in such a way that the combinations are chemically compatible and do not form complexes which precipitate from the finished product.

In certain exemplary embodiments, the presently disclosed compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

In certain exemplary embodiments, the presently disclosed compositions can include or more silicone containing compounds. By way of example and not limitation, silicone containing compounds can include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

Non-limiting examples of silicone-containing compounds that can be included include silicone oils (e.g., volatile and non-volatile oils), gels, and solids. In certain aspects, the silicone containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e., normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicone 7207 (Union Carbide Corp., Danbury, Conn.); low viscosity dimethicones, e.g., dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described, for example, in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the presently disclosed subject matter include those available from General Electric Co., Silicone Products Div., Waterford, N.Y. and SWS Silicones Div. of Stauffer Chemical Co., Adrian, Mich.

Thickening agents, including thickener or gelling agents, include substances which that can increase the viscosity of a composition can also be included in the instantly disclosed compositions. Thickeners includes those that can increase the viscosity of a composition without substantially modifying the efficacy of the active ingredient within the composition. Thickeners can also increase the stability of the compositions of the presently disclosed subject matter. In certain aspects of the presently disclosed subject matter, thickeners include hydrogenated polyisobutene or trihydroxystearin, or a mixture of both.

Non-limiting examples of additional thickening agents that can be used in the context of the presently disclosed subject matter include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B.F. Goodrich).

Non-limiting examples of crosslinked polyacrylate polymers include cationic and nonionic polymers. Non-limiting examples are described in U.S. Pat. Nos. 5,100.660; 4.849,484; 4,835,206; 4.628.078; and 4,599,379.

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxylmethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically, these polymers are ethers of C₁₀-C₃₀ straight or branched chain alcohols with hydroxyalkylcelluloses. Other useful polysaccharides include scleroglucans comprising a linear chain of 1-3 linked glucose units with a 1-6 linked glucose every three units.

Non-limiting examples of gums that can be used with the presently disclosed subject matter include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Non-limiting examples of preservatives that can, in exemplary embodiments, be included in the instantly disclosed compositions include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

Since the pH of the skin is 5.5, compositions for topical skin application (to avoid irritation) should preferably have a pH value of between 4.0 and 9.0, preferably between 5.0 and 8.0, a pH adjusting composition is typically added to bring the pH of the composition to the desired value. The compositions of the presently disclosed subject matter therefore can, in exemplary embodiments, be formulated to have a pH value that ranges between about 4.0 and about 9.0, more preferably between about 5.0 and about 8.0.

Suitable pH adjusting agents include, for example, but are not limited to, one or more adipic acids, glycines, citric acids, calcium hydroxides, magnesium aluminometasilicates, buffers or any combinations thereof.

The instantly disclosed compositions can, in certain embodiments, also include a penetration enhancer. A “penetration enhancer” is an agent known to accelerate the delivery of a substance through the skin. Suitable penetration enhancers usable in the presently disclosed subject matter include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond. Va.), alcohols, and the like. The permeation enhancer can also be a vegetable oil. Such oils include, for example, safflower oil, olive oil, coconut oil, cottonseed oil and corn oil.

In certain embodiments, the presently disclosed compositions can be incorporated in a medical device (e.g., a suture or skin engagement device) or medical apparatus (e.g., a bandage, adhesive, wrap or other material intended to be applied or implanted directly to the skin) as a combination product to be applied, injected or implanted into a subject. For example, a bandage can be impregnated with the presently disclosed compositions such that the presently disclosed compositions elute from the bandage into the skin of the subject over a period of time (e.g., a day, week, month, 3-months, or longer) as the bandage is worn by the subject. See, e.g., International Patent Application No. WO 2006/062756, hereby incorporated by reference. Similarly, an implantable medical device can be impregnated with a composition of the present application that is to be eluted over time according to known methods. See, e.g., International Patent Application No. WO 2011/119159, hereby incorporated by reference.

The compositions of the subject disclosure can be in a form suitable for oral use, for example, as tablets, troches, lozenges, aqueous or oily suspensions, solutions, dispersible powders or granules, emulsions, hard or soft capsules, syrups or elixirs, pastes, gels or the like. Compositions intended for oral use can be prepared according to any known method, and such compositions can contain one or more agents selected from the group consisting of sweetening agents, flavoring agents, coloring agents, and preserving agents in order to provide pharmaceutically elegant and palatable compositions. Tablets or other oral dosage forms can contain the active ingredient(s) in admixture with non-toxic pharmaceutically-acceptable excipients which are suitable for the manufacture of tablets or oral dosage form. These excipients can be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets or oral dosage forms can be uncoated or they can be coated by known techniques to delay disintegration and absorption in the gastrointestinal tract and thereby provide a sustained action over a longer period, such as by providing an enteric or gas-resistant coating. For example, a time delay material such as glyceryl monostearate or glyceryl distearate can be employed. They also can be coated for controlled delivery. For example, a “delayed release” dosage form releases a product or substance at a time other than promptly after administration. Examples of delayed-release systems include repeat-action tablets and capsules, and enteric-coated tablets where timed release is achieved by a barrier coating.

Compositions of the subject disclosure also can be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the subject disclosure can be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents can be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also can contain one or more coloring agents, one or more flavoring agents, and one or more sweetening agents, such as sucrose or saccharin.

Compositions of the subject disclosure can be formulated as oily suspensions by suspending the active ingredient in a vegetable oil, for example arachis oil, olive oil, sesame oil or coconut oil, or in a mineral oil, such as liquid paraffin. The oily suspensions can contain a thickening agent, for example, beeswax, hard paraffin or cetyl alcohol. Sweetening agents, such as those set forth above, and flavoring agents can be added to provide a palatable oral composition. These compositions can be preserved by the addition of an antioxidant such as ascorbic acid.

Compositions of the subject disclosure can be formulated in the form of dispersible powders and granules suitable for composition of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, or example, sweetening, flavoring and coloring agents also can be present.

The compositions of the presently disclosed subject matter also can be in the form of oil-in-water emulsions. The oily phase can be a vegetable oil, for example, olive oil or arachis oil, or a mineral oil, for example a liquid paraffin, or a mixture thereof. Suitable emulsifying agents can be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also can contain sweetening and flavoring agents.

The compositions of the presently disclosed subject matter also can be formulated as syrups and elixirs. Syrups and elixirs can be formulated with sweetening agents, for example, glycerol, propylene glycol, sorbitol or sucrose. Such formulations also can contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

Liquid based oral dosage forms, like their solid counterparts, can, in certain embodiments contain at least 0.1 mg of a provided composition. One skilled in the art will be able to properly formulate a liquid formulation containing an appropriate amount of a provided composition per fluidic ounce, depending on the additive or carrier selected.

Formulations suitable for buccal administration include tablets and lozenges comprising a composition of the subject disclosure in a flavored base, such as sucrose, acacia or tragacanth; and pastilles comprising the composition in an inert base, such as gelatin and glycerin or sucrose and acacia. Such formulations can, in certain embodiments, be formulated to adhere or reside in close proximity to the teeth, gums and other dental tissue according to methods known in the art.

In practical use, a provided composition of the subject disclosure can be combined as the active ingredient in an admixture with a pharmaceutical carrier according to conventional pharmaceutical compounding techniques. The carrier can take a wide variety of forms depending on the form of preparation desired for administration, for example, oral, parenteral (including intravenous), urethral, vaginal, nasal, dermal, transdermal, pulmonary, deep lung, inhalation, buccal, sublingual, or the like. In preparing the compositions for oral dosage form, any of the usual pharmaceutical media can be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets.

The compositions of the subject disclosure can be incorporated into all types of vehicles. Non-limiting examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use according to the subject disclosure. In certain aspects, it is important that the concentrations and combinations of the compounds, ingredients, and agents be selected in such a way that the combinations are chemically compatible and do not form complexes which precipitate from the finished product.

For parenteral administration (e.g., intravenous or intraperitoneal), the compositions of the subject disclosure can contain any pharmaceutically acceptable excipient compatible with the active agents and capable of providing the desired pharmacological release profile for the dosage form. Excipients include, for example, suspending agents, surfactants, solubilizers, stabilizers, lubricants, wetting agents, anti-foaming agent, diluents, and the like. Pharmaceutically acceptable excipients can comprise, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

Suitable antifoaming agents include dimethicone, myristic acid, palmitic acid, and simethicone.

The compositions of the present application can also contain a non-aqueous diluent such as ethanol, one or more polyol (e.g. glycerol, propylene glycol), an oil carrier, or any combination of the foregoing.

The compositions of the present application can additionally comprise a preservative. The preservative may be used to inhibit bacterial growth or prevent deterioration of the active agent. Preservatives suitable for parenteral formulations include ascorbic acid, acetylcysteine, benzalkonium chloride, benzethonium chloride, benzoic acid, benzyl alcohol, chlorbutanol, chlorhexidene, m-cresol, 2-ethoxyethanol, human serum albumin, monothioglycerol, parabens (methyl, ethyl, propyl, butyl, and combinations), phenol, phenylmercurate salts (acetate, borate nitrate), sorbic acid, sulfurous acid salts (bisulfite and metabisulfite), and thimerosal. In certain embodiments the preservative is an antioxidant such ascorbic acid, glutathione, or an amino acid. Amino acids useful as antioxidants include methionine, cysteine, and L-arginine.

For intragastric administration (e.g., via gastrostomy or oral gavage), oral formulations can be modified to facilitate delivery of the drug within the stomach. See. e.g., Susan Hua, Advances in Oral Delivery for Regional Targeting in the Gastrointestinal Tract—Influence of Physiological, Pathophysiological and Pharmaceutical Factors, Front Pharmacol. 2020; 11:524. For example, in situ gel systems can be employed allow the formulation to float or reside in the stomach for extended periods of time, and can find use according to the subject disclosure. See, e.g., Bhardwaj, Lovenish et al., A Short Review on Gastro Retentive Formulations for Stomach Specific Drug Delivery: Special Emphasis on Floating In situ Gel Systems, African Journal of Basic and Applied Sciences (2011).

The following examples are included to demonstrate certain non-limiting aspects of the presently disclosed subject matter. It should be appreciated by those of skill in the art that the techniques disclosed in the examples which follow represent techniques discovered by the inventor to function well in the practice of the presently disclosed subject matter. However, those of skill in the art should, in light of the present disclosure, appreciate that many changes can be made in the specific embodiments which are disclosed and still obtain a like or similar result without departing from the spirit and scope of the presently disclosed subject matter.

### EXAMPLES

### Example 1: Bioinformatics Analysis

A library of food derived small molecules was screened to simulate binding affinities to HIF prolyl hydroxylase 2 (PHD2/EGLN1), as compared to the known inhibitor Vadadustat, which has an affinity of −7.3. The following molecules were identified as having a binding affinity to HIF prolyl hydroxylase 2 (PHD2/EGLN1):

TABLE 1
Binding affinities of the food-derived PHD enzyme inhibitors: The
affinities are high and comparable to known drug inhibitor (Vadadustat
with an affinity of −7.3; https://www.rcsb.org/structure/5OX6).

| Molecule Name | CAS Number | Binding affinity |
|---|---|---|
| Glabridin | 59870-68-7 | −7.9 |
| Puerarin | 3681-99-0 | −8.7 |
| Wedelolactone | 524-12-9 | −8.6 |
| Phlorizin | 60-81-1 | −6.8 |
| Salvianolic acid B | 121521-90-2 | −8.1 |
| Angoroside C | 115909-22-3 | −7.9 |

A library of food derived small molecules was analyzed to simulate binding affinities to von Hippel Lindau protein, as compared to the known inhibitor ZINC95921109, which has an affinity of −6.8. The following molecules were identified as having a binding affinity to VHLP:

TABLE 2
Binding affinities of the food-derived VHL inhibitors: The affinities
are high and comparable to known drug inhibitor (ZINC95921109
with an affinity of -6.8; https://www.rcsb.org/structure/4BKT).

| Molecule Name | CAS Number | Binding affinity |
|---|---|---|
| Meisoindigo | 97207-47-1 | −7.3 |
| Tanshinone IIA | 568-72-9 | −6.9 |
| 7-Hydroxyflavone | 6665-86-7 | −6.5 |
| Dicumarol | 66-76-2 | −6.5 |
| Flavone | 525-82-6 | −6.3 |
| Tabersonine | 4429-63-4 | −6.2 |
| Danthron | 117-10-2 | −6.1 |
| Wedelolactone | 524-12-9 | −6.1 |
| Equol | 531-95-3 | −6 |

### Example 2: Molecular Docking Analysis

A 5OX6 HIF prolyl hydroxylase 2 (PHD2/EGLN1) in complex with vadadustat, a known inhibitor is analyzed. See Protein Data Bank “5OX6” at www.rcb.org/structure/5ox6 and Chem. Sci. 2017, 8, 7651 (2017). Structure key residues or drug pocket locations were identified, including hydrogen bonds with the key residues of His 313 and Arg 383 on the PHD2 protein domain. Hydrogen bonds suggest critical areas to target the inhibitory molecules as they will compete with the substrate molecule (HIF), additionally, this confers higher affinity and better effectiveness and specificity.

A library of food derived small molecules were screened against these key residues using mcule.com and Lea3D (https://chemoinfo.ipmc.cnrs.frALEA3D/index.html) modelling interfaces. Flavone, along with the other ARG-383 targeting components listed in Table 1, was found to have a high affinity interacting with Arg-383. Puerarin, along with the other HIS 3-13 targeting components, was found to have a high affinity interacting with His-313.

TABLE 3
ARG-383 targeting component

| Name | CAS Number |
|---|---|
| Flavone | 525-82-6 |
| Meisoindigo | 97207-47-1 |
| Echinatin | 34221-41-5 |
| Danthron | 117-10-2 |
| Eriodictyol | 552-58-9 |
| Caffeic acid phenethyl ester | 104594-70-9 |
| Myricetin | 529-44-2 |
| Xanthotoxol | 2009-24-7 |

TABLE 4
HIS-313 targeting component

| Name | CAS Number |
|---|---|
| Puerarin | 3681-99-0 |
| Glabridin | 59870-68-7 |
| Wedelolactone | 524-12-9 |
| Meisoindigo | 97207-47-1 |
| Sophoricoside | 152-95-4 |
| Isovitexin | 38953-85-4 |
| Phlorizin | 60-81-1 |

### Example 3: In Vitro HIF Stabilization

L929 cells (Sigma Aldrich Catalog No. 8511425-1VL; Lot No. 18D043) were placed at 10,000 cells/cm² in 6 well plates. 10 mM CoCl₂, 10 mM Flavone (Manufacturer: Selleckchem Catalog: S3967 Lot: S396701), and 10 mM Puerarin (Manufacturer: Selleckchem Catalog: S2346 Lot: 01) stock solution in DMSO were prepared. Serial dilutions from 10 mM stock into 100% DMSO to create 1000 uM stock, 100 uM stock and 10 um stock and 1 um stock were performed. Twenty-three hours after plating, media was prepared by adding 200 μL of each stock and control to 19.8 mL of complete media. Twenty-four hours after plating, media was replaced with treated media. After 2 hours, 4 hours, 16 hours, 24 hours and 48 hours, cells were harvested by washing with PBS then the cells were lysed using 1× lysis buffer provided from AbCam ELISA kit. Pierce mini protease tablets were added as an extra precaution against protein degradation. HIF1-a protein concentration was measured by ELISA according to the manufacturer protocol. Total protein concentration was measured using the Detergent Compatible Bradford assay. DNA content was measured by DNA Quantification Assay kit.

The CoCl₂ positive control (200 micromolar) shows a 2.21× and 2.16× fold HIF content increase (normalized to the DNA content) when compared to the negative control, confirming that the methodology and analysis were valid and within the measurable range.

Flavone and Puerarin alone (does of 0.01, 0.1, 1, 10, and 100 micromolar) show either a decrease of HIF-1a or the same level of HIF-1a when compared to the negative control cells.

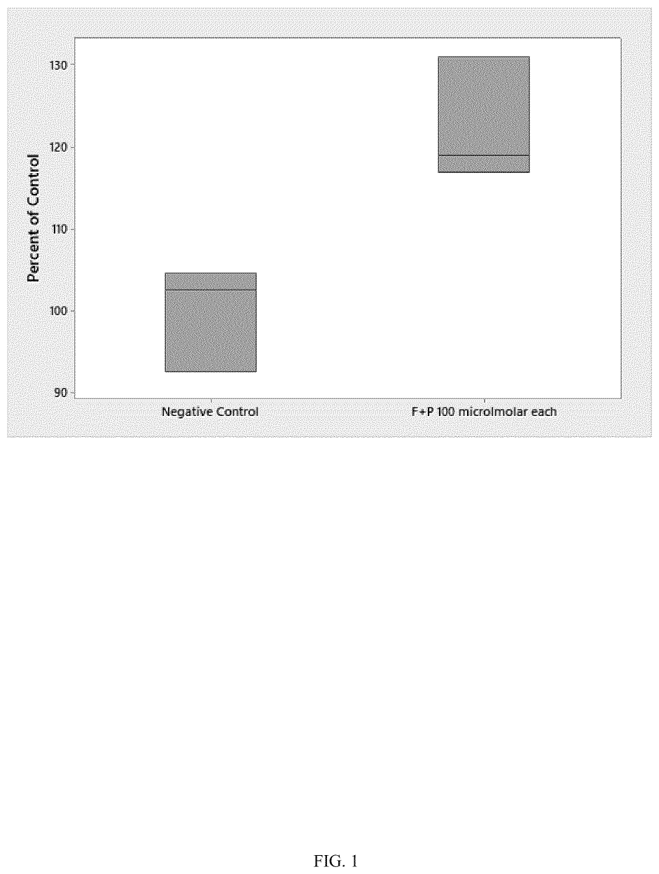

HIF content normalized to DNA content shows that only the combination of Flavone and Puerarin at 100 uM increases the level of HIF-1a by 1.22× fold as compared to the negative control cells, as shown in FIG. 1. FIG. 1 is a boxplot displaying HIF content data as a percent of the negative control. Flavone+Puerarin (dose of 100 micromolar each) treated cells had statistically significant (P-value: 0.017) higher (˜22%) HIF levels when compared to the negative control.

### Example 4: In Vivo Mice Testing

Flavone (>98% purity) was obtained from Tokyo Chemical Industry Co., Ltd. (CAS RN: 525-82-6; Lot No. YSFOD-HN) and puerarin (≥98% purity) was obtained from ADAMAS-BETA (Lot No. P1342643). Eucerin® cream was obtained from Beiersdorf AG (Lot No. 91429257). Corn oil was obtained from MedChemExpress Co., Ltd. (Catalogue Number HY-Y1888/CS-0040437: Lot No. 66633). DMSO was obtained from Sinopharm Chemical Reagent Co., Ltd. (Catalogue Number 30072418; Lot No. 20190612).

Stock solutions were prepared as follows:
1. Solution A: Mix 100 microliter DMSO with 1 gram of flavone:
2. Solution B: Mix 100 microliter DMSO with 1 gram of puerarin;
3. Solution C: Mix solution A and B;
4. Solution D: Add 50 ml of corn oil to solution D.

Testing preparations were introduced to a Eucerin® cream carrier as follows:
Preparation A: 2.0%: 0.5 ml of solution D+1 gram of the carrier:
Preparation B: 1.0%: 0.25 ml of solution D+1 gram of the carrier,
Preparation C: 0.5%: 0.12 ml of solution D+1 gram of the carrier;

A total of 24 (4 for backup) male naïve db/db mice, weighing 20-25 grams were obtained from GemPharmatech Co., pathogen free and approximately 6-7 weeks old upon arrival. The diabetic mice were adapted for eight days in a SPF room at 20-26° C. at 40-70% RH with a 12-hour fluorescent light/dark cycle, 2-5 mice housed per cage by treatment group. Access to food and water was provided ad libitum.

Animals were assigned to treatment groups by randomization in BioBook software to achieve similar group mean body weight, which provides control of bias, and dosed as follows:

| | | | | | Dosage | |
|---|---|---|---|---|---|---|
| Group | Test Article | No. | Route | Conc. | mg/kg | Regimen |
| 1 | db/db + Carrier | 5 | painted | N/A | 50 mg | Once daily for 21 |
| 2 | db/db + test | 5 | painted | 0.5% | 50 mg | days |
| | article low dose | | | | | |
| | (Preparation C) | | | | | |
| 3 | db/db + test | 5 | painted | 1.0% | 50 mg | |
| | article middle | | | | | |
| | dose (Preparation B) | | | | | |
| 4 | db/db + test | 5 | painted | 2.0% | 50 mg | |
| | article high dose | | | | | |
| | (Preparation A) | | | | | |

To model a full-thickness diabetic wound, the dorsa of animal subjects were depilated on the day of wounding and a critical and full-thickness excisional wound of 1 cm×1 cm square was made using a template. One wound was cut per animal. A small ruler was placed in the plane of the wound, and images were taken of the wounds using a digital camera. Wounds were imaged at each of these time points in a similar manner to the day of wounding and quantified using Image J software. Each post-wounding measurement was normalized to its corresponding day 0 wound area and recorded as a percentage of the original wound remaining open. During the course of treatment, no systemic adverse events were observed, with observations limited to local swelling.

The following results were obtained:

| | | Open Wound Area (% of Original) | | | | | | | | | |
|---|---|---|---|---|---|---|---|---|---|---|---|
| Group | | Day 2 | Day 4 | Day 6 | Day 8 | Day 10 | Day 12 | Day 14 | Day 16 | Day 18 | Day 21 |
| G1: Vehicle, | Mean | 118.61 | 113.51 | 132.61 | 142.89 | 80.99 | 43.56 | 11.77 | 10.00 | 6.99 | 0.53 |
| 50 mg, qd | SEM | 3.63 | 3.45 | 2.71 | 9.15 | 3.12 | 7.64 | 1.08 | 1.65 | 2.78 | 0.53 |
| G2: Test article | Mean | 135.56 | 126.10 | 124.21 | 123.02 | 68.34 | 40.84 | 9.64 | 8.35 | 4.10 | 0.00 |
| 0.5%, 50 mg, qd | SEM | 1.98 | 9.22 | 10.83 | 9.80 | 7.25 | 10.15 | 2.65 | 3.38 | 2.62 | 0.00 |
| G3: Test article | Mean | 102.44 | 109.67 | 118.60 | 122.05 | 95.46 | 59.37 | 39.24 | 22.49 | 9.93 | 1.81 |
| 1.0%, 50 mg, qd | SEM | 3.81 | 5.15 | 4.54 | 6.98 | 4.16 | 2.78 | 4.99 | 7.12 | 3.09 | 0.96 |
| G4: Test article | Mean | 104.33 | 114.76 | 118.82 | 107.64 | 82.09 | 52.02 | 30.25 | 15.10 | 5.46 | 1.19 |
| 2.0%, 50 mg, qd | SEM | 9.59 | 12.89 | 14.12 | 8.48 | 9.19 | 4.48 | 7.04 | 2.37 | 2.16 | 0.51 |

| | Open Wound Area (cm²) | | | | | | | | | | | |
|---|---|---|---|---|---|---|---|---|---|---|---|---|
| Group | | Day 0 | Day 2 | Day 4 | Day 6 | Day 8 | Day 10 | Day 12 | Day 14 | Day 16 | Day 18 | Day 21 |
| G1: Vehicle, | Mean | 1.28 | 1.52 | 1.46 | 1.70 | 1.81 | 1.03 | 0.56 | 0.15 | 0.13 | 0.09 | 0.01 |
| 50 mg, qd | SEM | 0.05 | 0.06 | 0.09 | 0.07 | 0.08 | 0.04 | 0.10 | 0.01 | 0.02 | 0.04 | 0.01 |
| G2: Test article | Mean | 1.37 | 1.86 | 1.72 | 1.70 | 1.67 | 0.93 | 0.55 | 0.13 | 0.11 | 0.05 | 0.00 |
| 0.5%, 50 mg, qd | SEM | 0.05 | 0.05 | 0.11 | 0.14 | 0.10 | 0.09 | 0.13 | 0.03 | 0.04 | 0.04 | 0.00 |
| G3: Test article | Mean | 1.40 | 1.43 | 1.52 | 1.66 | 1.70 | 1.33 | 0.83 | 0.55 | 0.32 | 0.14 | 0.03 |
| 1.0%, 50 mg, qd | SEM | 0.06 | 0.08 | 0.04 | 0.09 | 0.11 | 0.07 | 0.07 | 0.07 | 0.10 | 0.05 | 0.01 |
| G4: Test article | Mean | 1.44 | 1.48 | 1.63 | 1.68 | 1.53 | 1.16 | 0.74 | 0.42 | 0.21 | 0.08 | 0.02 |
| 2.0%, 50 mg, qd | SEM | 0.05 | 0.09 | 0.14 | 0.15 | 0.08 | 0.10 | 0.05 | 0.08 | 0.03 | 0.03 | 0.01 |

Animals in all groups healed over 90% of their original dermal wound areas by day 18 of the experiment. The open wound area of diabetic mice treated with test article 2.0% decreased significantly on day 8 compared to diabetic mice treated with Vehicle. However, it is also observed that open wound area increased significantly on day 14 in diabetic mice treated with test article 1% and 2%.

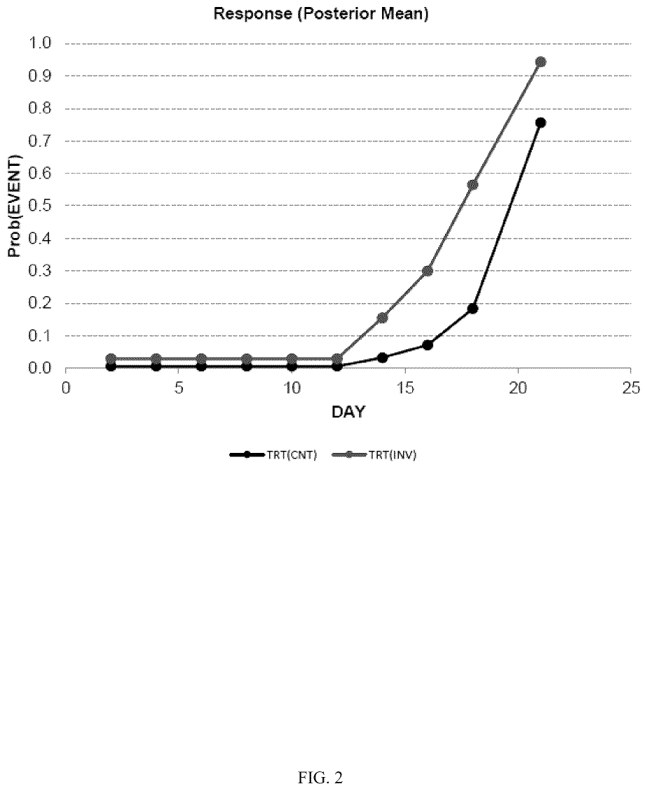

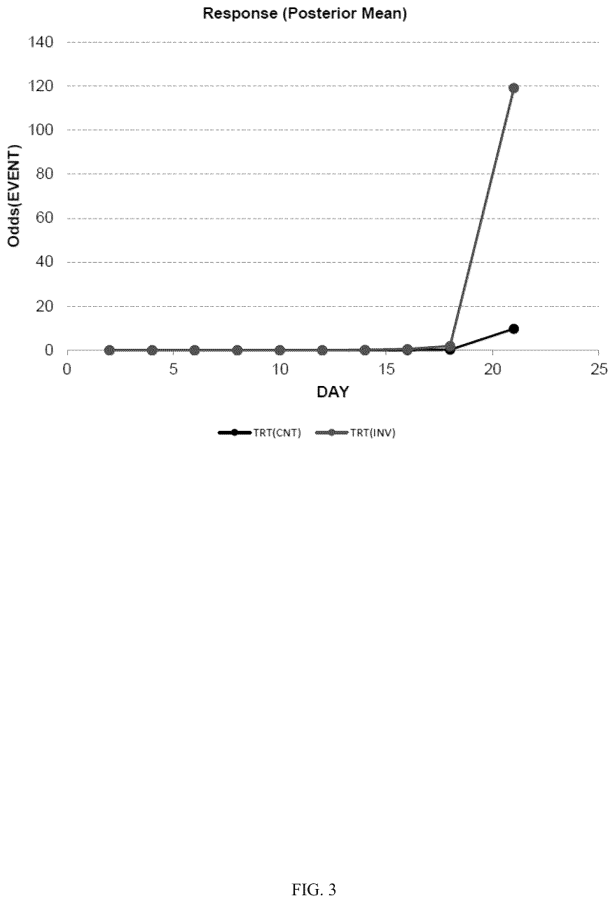

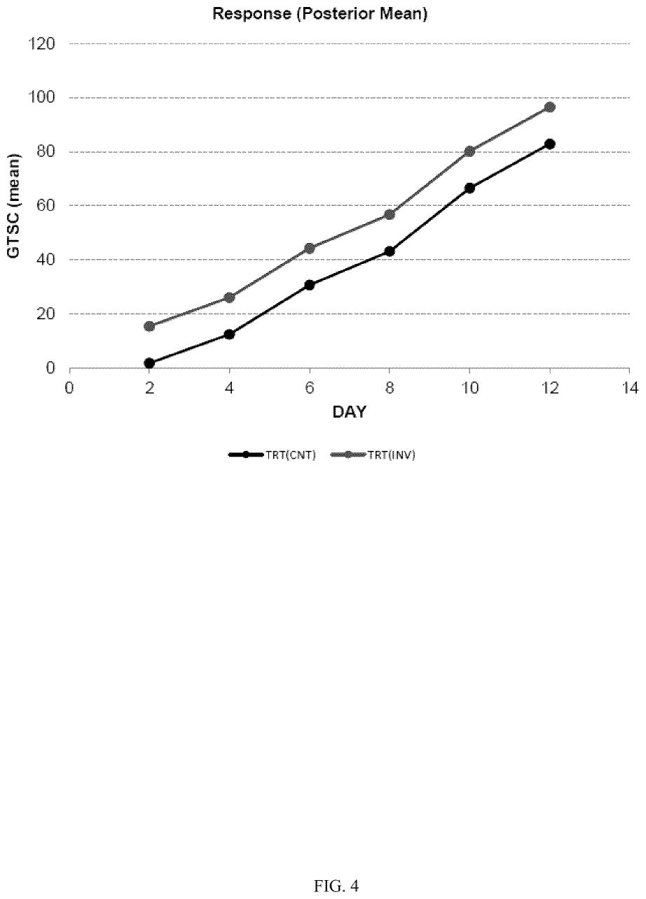

The dosing strategy in this Example was designed to support selection based on the lowest effective dose (LED). The data showed that the dose of 0.5% concentration is the LED with sufficient effectiveness to accelerate complete wound closure (CWC) without adverse events. Therefore, a further analyses was conducted comparing this dose to the control group. Further analyses included probability, and odds of CWC at different time points (FIGS. 2 and 3) and granulation tissue formation (GTF) (FIG. 4).

Regarding GTF, the methodology of Valenzuela-silva et al., Diabetes Care, 2013 February; 36(2):210-215 was used. The data showed that from day 2 to 14, there were consistently higher percentages of GTF in wounds treated with 0.5% test articles (INV) when compared to the vehicle-treated test articles (CNT) (FIG. 4). The 0.5% test articles (INV) is the line shown on the top of FIG. 4, and the vehicle-treated articles (CNT) is the line shown on the bottom of FIG. 4.

Regarding the probability of CWC at different timepoint. CWC was defined as a wound size of ≤0.01. The data showed consistently higher percentages of CWC in wounds treated with 0.5% test articles (INV) when compared to the vehicle-treated test articles (CNT) (FIG. 2). On day 21, the odds of CWC were 10-fold higher in wounds treated with 0.5% test articles when compared to the vehicle-treated test articles (FIG. 3). The 0.5% test articles (INV) are the lines shown on the top of FIGS. 2 and 3, and the vehicle-treated articles (CNT) are the lines shown on the bottom of FIGS. 2 and 3.

### Example 5: Clinical Case Studies

Bioinformatic, in vitro, and animal data suggested that the use of the flavone-puerarin combination can promote HIF stabilization which can benefit the process of wound healing. Therefore, administration of a cream preparation containing puerarin (0.25 wt %) and flavone (0.25 wt %) was manufactured and tested in this clinical cases series. The cream was administered at least once daily in an amount sufficient to cover the wound area.

The patient's age, gender, medical history, and clinical data were collected in a case series study design. Clinical data included TTH (time-to-heal), wound pictures, pain, and adverse events. TTH is defined as the time between the first application of the product until healing success. Healing success was defined as the complete closure of the wound.

A total of 8 cases was analyzed (3 due to injury and 5 surgical wounds). Six females and 2 males with the age range of 32 to 85 years old with a median age of 58 and a mean of 60 years. The majority of the cases had risk factors including the elderly (5 out of 8), diabetes (5 out of 8), hypertension (3 out of 8), and hyperlipidemia (2 out of 8). A single case may have more than one risk factor.

All cases had successful healing with short TTH, observed pain reduction, and with no adverse events. TTH ranged from 3 to 22 days with a median of 14.5 and a mean of 14.9 days.

### Discussion

Chronic wounds represent a major medical, social, and economic problem. As no efficient therapy is available, it is a high priority to develop new strategies for the treatment of this devastating complication. The pathogenesis of chronic wounds is still unclear.

A critical stimulus for normal wound healing is relative hypoxia, and an impaired reaction to hypoxia could contribute to impaired wound healing. Hypoxia has been shown to induce major cytokines (VEGF, TGF-b, PDGF), to stimulate the proliferation and migration of fibroblasts and keratinocytes.

Cellular adaptive responses to hypoxia are mediated by the hypoxia-inducible factor (HIF). Regulation of HIF-1 activity is dependent on the degradation of the HIF-1 subunit in normoxia. The molecular basis of its degradation is O-dependent hydroxylation of at least one of the two proline residues in the oxygen-dependent degradation domain of HIF-1 by specific Fe2- and oxoglutarate-dependent prolyl 4-hydroxylases (PHDs). In its hydroxylated form, HIF-1 binds to the von Hippel-Lindau (VHL) tumor suppressor protein that is part of an E3 ubiquitin ligase complex targeting HIF-1 for proteasomal degradation.

Under hypoxic conditions, HIF-1 is stabilized against degradation and transactivates and up-regulates a series of genes that enable cells to adapt to reduced oxygen availability. HIF-1 plays a pivotal role in wound healing, and its expression in the multistage process of normal wound healing has been demonstrated. In essence, HIF-1 is necessary for the expression of multiple angiogenic growth factors, cell motility, and the recruitment of endothelial progenitor cells. Several studies had shown that hyperglycemia impairs HIF-1 stability and function, and low levels of HIF-1 expression in foot ulcer biopsies in patients with diabetes was demonstrated here.

In this clinical case series, there were five female cases with diabetes (three injuries and two surgical wounds) with a median age of 45. All cases had successful healing with no adverse events. TTH was short for all cases with a median of 18 days.

In conclusion, targeting HIF-1 has several important clinical implications for tissue repair, especially for cases with risk factors.

### Example 6: Topical Cream Formulation

A cream base is prepared from water, petrolatum, mineral oil, ceresin, lanolin alcohol, phenoxyethanol and piroctone olamine. To the cream base, the following components in the amounts shown below (w/w) can be mixed to form a formulation suitable for topical administration to a human.

| | |
|---|---|
| Cream Base | 87.5% |
| Flavone | 0.25% |
| Puerarin | 0.25% |
| corn oil | 12.0% |
| DMSO | >.01% |
| Total | 100% |

### Example 7: Topical Gel Formulation

A gel base is prepared from water, vegetable glycerin, PEG 400, and PEG 3350 in the amounts shown below. To the gel base, the following components in the amounts shown below (w/w) can be mixed to form a formulation suitable for topical administration to a human.

| | |
|---|---|
| Distilled Water | 39.5 |
| Meadowsweet Extract | 4 |
| Oak Extract | 4 |
| Vegetable Glycerin | 2.3 |
| Polyethylene Glycol 400 | 5 |
| Polyethylene Glycol 3350 | 5 |
| Zinc Acetate | 0.8 |
| Flavone | 0.25 |
| Puerarin | 0.25 |
| Shea Butter | 12 |
| Sesame Oil | 15 |
| Gelmaker ® EMU | 2.2 |
| Emulsifying Wax NF | 4.6 |
| Polysorbate 80 | 4.5 |
| Phenoxyethanol | 0.6 |
| Total | 100% |

### Example 8: Topical Formulation for Burn Relief

An ointment base is prepared from water, petroleum jelly, sesame oil and shea and olive butter in the amounts shown below. To the base, the following components in the amounts shown below (w/w) can be mixed to form a formulation suitable for topical administration to a human.

| | |
|---|---|
| Distilled Water | 35 |
| Flavone | 0.25 |
| Petroleum Jelly | 1.5 |
| Puerarin | 0.25 |
| ß-Sitosterol | 0.25 |
| Aloe Vera Juice | 4.5 |
| Sesame Oil | 12 |
| Almond Oil | 6 |
| Chamomile Oil | 4.25 |
| Grape Seed Oil | 4 |
| Shea Butter | 10 |
| Olive Butter | 8 |
| Bees Wax | 8 |
| Polysorbate 80 | 4.8 |
| Vitamin B3 | 0.4 |
| Lavender E. Oil | 0.5 |
| Optiphen ™ | 0.3 |
| Total | 100% |

### Example 9: Topical Formulation for Pet Wounds

An ointment base is prepared from water, glycerin, coconut oil, olive oil and grapeseed oil in the amounts shown below. To the base, the following components in the amounts shown below (w/w) can be mixed to form a formulation suitable for topical administration to a human.

| | |
|---|---|
| Distilled Water | 38.5 |
| Black Coffee Extract | 2.5 |
| Vegetable Glycerin | 2.4 |
| Guar Gum | 0.8 |
| Orange Peel Extract | 2.8 |
| Papaya Extract | 2.2 |
| Puerarin | 0.25 |
| Vitex Castus extact | 0.25 |
| Marshmallow Root Extract | 3 |
| Candelilla Wax | 12 |
| Emulsifying Wax NF | 10 |
| Coconut Oil | 6 |
| Olive Oil | 6 |
| Grape Seed Oil | 6.5 |
| Polysorbate 80 | 4.8 |
| Lavender Essential Oil | 1 |
| Phenoxyethanol | 0.8 |
| Citric Acid | 0.2 |
| Total | 100% |

All publications, patent and patent applications mentioned in this specification are incorporated herein by reference to the same extent as if each individual publication, patent or patent application was specifically and individually incorporated by reference.

While the presently disclosed subject matter has been particularly shown and described with references to preferred embodiments thereof, it will be understood by those skilled in the art that various changes in form and details can be made therein without departing from the scope of the presently disclosed subject matter, as encompassed by the appended claims. Further, all embodiments included herein are given solely for the purpose of illustration and are not to be construed as limitations of the presently disclosed subject matter, as many variations thereof are possible without departing from the spirit and scope of the presently disclosed subject matter.

1. A composition for administration to a subject comprising (a) an effective amount of a von Hippel Lindau protein inhibitor selected from meisoindigo, tanshinone IIA, 7-hydroxyflavone, dicumarol, flavone, tabersonine, danthron, equol, and pharmaceutically acceptable salts of each of the foregoing, and (b) an effective amount of a PHD2/EGLN1 protein inhibitor selected from glabridin, puerarin, wedelolactone, phlorizin and pharmaceutically acceptable salts of each the foregoing.

2. The composition of claim 1, wherein the von Hippel Lindau protein inhibitor is flavone.

3. The composition of claim 2, wherein the PHD2/EGLN1 protein inhibitor is puerarin.

4. The composition of claim 1, wherein the PHD2/EGLN1 protein inhibitor is puerarin.

5. The composition of claim 1, wherein the weight ratio of von Hippel Lindau protein inhibitor: PHD2/EGLN1 protein inhibitor is from about 0.01 to about 100.

6. The composition of claim 1, wherein the weight ratio of von Hippel Lindau protein inhibitor: PHD2/EGLN1 protein inhibitor is from about 0.01 to about 100.

7. The composition of claim 6, wherein the weight ratio weight ratio of von Hippel Lindau protein inhibitor: PHD2/EGLN1 protein inhibitor is from about 0.01 to about 0.5.

8. The composition of claim 6, wherein the weight ratio weight ratio of von Hippel Lindau protein inhibitor: PHD2/EGLN1 protein inhibitor is from about 0.5 to about 1.5.

9. The composition of claim 3, wherein the weight ratio of flavone:puerarin is from about 0.01 to about 100.

10. The composition of claim 9, wherein the weight ratio weight ratio of flavone:puerarin is from about 0.01 to about 0.5.

11. The composition of claim 9, wherein the weight ratio weight ratio of flavone:puerarin is from about 0.5 to about 1.5.

12. A composition for administration to a subject comprising an ARG-383 targeting component selected from flavone, echinatin, danthron, eriodictyol, caffeic acid phenethyl ester, myricetin, xanthotoxol, and pharmaceutically acceptable salts of each the foregoing; and a HIS-313 targeting component selected from puerarin, glabridin, wedelolactone, meisoindigo, sophoricoside, isovitexin, phlorizin, and pharmaceutically acceptable salts of each of the foregoing.

13. The composition of claim 12, wherein the ARG-383 targeting compound is flavone.

14. The composition of claim 12, wherein the HIS-313 targeting compound is puerarin.

15. The composition of claim 1, further comprising a carrier.

16. The composition of claim 15, wherein the carrier includes one or more of petrolatum, mineral oil, corn oil, vegetable oil, glycerin, polyethylene glycol, sesame oil, coconut oil, olive oil, grapeseed oil, shea butter and olive butter.

17. The composition of claim 1, further comprising a 5 skin penetration enhancer.

18. The composition of claim 1, wherein the composition is in a form suitable for topical administration to the skin of a human.

19.-38. (canceled)
